(19) United States
(12) Patent Application Publication
(10) Pub. No.: US 2020/0255807 A1
(43) Pub. Date: Aug. 13, 2020

(54) IN VITRO PROPAGATION OF PRIMARY CANCER CELLS

(71) Applicant: University of Cape Town, Cape Town (ZA)

(72) Inventors: Keertan Unkha Jairam DHEDA, Cape Town (ZA); Michele TOMASICCHIO, Cape Town (ZA)

(21) Appl. No.: 16/479,202

(22) PCT Filed: Jan. 19, 2018
(86) PCT No.: PCT/IB2018/050341

§ 371 Date: Jul. 18, 2019

(30) Foreign Application Priority Data
Jan. 19, 2017 (GB) 1700952.3

(51) Int. Cl. C12N 5/09

(52) U.S. Cl. CPC C12N 5/0693; C12N 2501/392; C12N 2501/11; C12N 2501/999; C12N 2501/39; C12N 2501/73; C12N 2500/24; C12N 2500/10; C12N 2500/30; C12N 2500/00; C12N 2501/33

(57) ABSTRACT

The invention relates to an in vitro method of obtaining and culturing primary tumour cells from a tissue sample using an isolation buffer, which includes collagenase II and optionally hyaluronidase and a propagation medium which includes estradiol or EGF. The invention also relates to a kit for obtaining and culturing primary tumour cells.

This application is a U.S. national phase submitted under 35 U.S.C. 371 of and claims priority to International Application No. PCT/IB2018/050341, which was filed on Jan. 19, 2018, which claims priority to and benefit of application GB 1700952.3, filed on Jan. 19, 2017. The contents of each of the above-referenced application are hereby expressly incorporated by reference in their entirety.

### BACKGROUND OF THE INVENTION

The isolation and propagation (long term growth in culture) of primary tumour cells remains problematic. Primary tumour cells are difficult to isolate and given their fastidious growth and survival requirements, they only survive for a few days in in vitro culture. This subverts optimal vaccine and drug development especially when autologous (patient specific) responses are required using cellular vaccines.

Dendritic and T-cell vaccines have shown tremendous promise against cancer, which remains the largest global killer of mankind. Dendritic cell vaccines (DCVs) have targeted several cancers, and there are currently a number of clinical trials involving the use of dendritic cells to treat various cancers. Development of a primary cancer cell model will accelerate the development of immunotherapeutic interventions for cancers. However, optimal generation of effective cell based vaccines requires in vitro ‘education’ using the individuals own tumour cells.

The ability to culture primary tumour cells in vitro is important in vaccine and drug development because they closely approximate an in vivo model system. Immortalized cell lines have been used extensively in cancer biology and as model system to test the efficacy of DCVs, but their use is becoming increasingly limited because of extensive genetic and phenotypic variations in people compared to those expressed in cell lines. The use of cell lines in cancer vaccine development is also a disadvantage because cell lines and primary cancer cells express antigens that are often different from those expressed on the tumours of individual patients. Therefore they remain poor “targets” in in vitro proof of concept cellular vaccine studies using dendritic cells and T-cell vaccines. In lay terms vaccines targeting the antigens expressed on tumour cell lines will not be effective in individual patients because their tumours display a varied and different repertoire of antigens (compared to those expressed in cell lines). There is thus an urgent need to develop new model systems to test the efficacy of dendritic and T-cell vaccines which are more representative of an in vivo model.

Based on the current literature, long term propagation of primary cancer cells from individual patients has proven nearly impossible. Thus, there is a need for an effective method of isolating and propagating primary tumour cells from biopsies for a prolonged period of time in vitro.

### SUMMARY OF THE INVENTION

The present invention relates to in vitro methods of obtaining and culturing primary tumour cells from a tissue sample using an isolation buffer which includes collagenase II and propagating the cells in a first propagation medium which includes estradiol or epithelial growth factor (EGF) and ethanolamine. The invention also relates to a kit for obtaining and culturing primary tumour cells.

In a first aspect of the invention there is provided for an in vitro method of obtaining and culturing primary tumour cells from a tissue sample, the method comprising the steps of firstly isolating the primary tumour cells using an isolation buffer which includes collagenase II for a period of 45 minutes or less at about 37° C., and secondly propagating the cells obtained in the isolation step using a first propagation medium which includes estradiol or EGF and ethanolamine.

It will be appreciated that the propagation medium of the invention may also include growth factors, supplements, steroids, antibiotics and hormones. Those of skill in the art will appreciate that growth factors may include but are not limited to activin, colony stimulating factor, epidermal growth factor, fibroblast growth factor, insulin-like growth factor, stem cell factor and/or transforming growth factor; the supplements may include but are not limited to amino acids and/or carbon sources; the steroids may include but are not limited to dexamethasone, prednisolone and/or testosterone; the antibiotics may include but are not limited to penicillin, streptomycin, kanamycin, tetracycline and/or gentamycin; and the hormones may include but are not limited to insulin, progesterone, luteinizing hormone, adrenalin, glucagon, oxytocin, thyroxine and/or vasopressin.

The in vitro method may further comprise a step of propagating the cells in a second propagation medium, wherein if the first propagation medium included estradiol, then the second propagation medium includes cortisol, and does not include estradiol. Alternatively, if the first propagation medium included EGF, then the second propagation medium includes cortisol and EGF.

In one embodiment of the invention the first propagation medium and second propagation medium include DMEM/F12, human A/B serum, an antibiotic, sodium pyruvate, insulin, transferrin, and/or selenium.

In another embodiment of the invention the first propagation medium and the second propagation medium may optionally include hyaluronidase, dispase II or papain. Preferably, the first propagation medium and the second propagation medium include hyaluronidase.

Preferably, the method also comprises a step of splitting the cells using trypsin.

In a preferred embodiment of the invention the tissue sample is obtained from a tumour biopsy. Preferably, the tissue sample is obtained from a human.

It will be appreciated that the tumour biopsy is from a cancerous tumour, wherein the cancer is selected from the group consisting of adrenal cancer including adrenocortical carcinoma and pheochromocytoma; anal cancer; appendix cancer; bile duct cancer including cholangiocarcinoma, extrahepatic bile duct cancer and intrahepatic bile duct cancer; bladder cancer including ureteral cancer; bone cancer including chondrosarcoma, Ewing sarcoma, osteogenic sarcoma, osteosarcoma, mesenchymal chondrosarcoma and bone sarcoma; brain cancer including anaplastic astrocytoma, astrocytoma, brain stem glioma, brain tumour, craniopharyngioma, diffuse astrocytoma, ependymoma, germ cell tumour, glioblastoma multiforme, glioma, low-grade astrocytoma, medulloblastoma, meningioma, mixed gliomas, oligodendroglioma, peripheral nerve cancer, pilocytic astrocytoma, pineal region tumour and pituitary gland cancer; breast cancer including ductal carcinoma in situ, male breast cancer, medullary carcinoma, infiltrating ductal carcinoma, infiltrating lobular carcinoma, inflammatory breast cancer, invasive or infiltrating breast cancer, lobular carcinoma in situ, metastatic breast cancer, mucinous carcinoma, Paget's disease, papillary carcinoma, triple-negative breast cancer and tubular carcinoma; cervical cancer; colorectal cancer including bowel cancer, colon cancer and rectal cancer; oesophageal cancer; eye cancer; gallbladder cancer; gastrointestinal cancer including gastrointestinal carcinoid cancer and gastrointestinal stromal tumours; head and neck cancer including neck cancer, tonsil cancer and metastatic squamous neck cancer; hemangioendothelioma; Hodgkin lymphoma including Hodgkin's disease; intestinal cancer; kidney cancer including renal cell carcinoma, renal pelvis cancer and ureteral cancer; leptomeningeal metastases; leukaemia including acute granulocytic leukaemia, acute lymphocytic leukaemia, acute myelogenous leukaemia, chronic lymphocytic leukaemia, chronic myelogenous leukaemia, hairy cell leukaemia and myelodysplastic syndrome; liver cancer; lung cancer including adenocarcinoma, adenosarcoma, small cell lung cancer, non-small cell lung cancer and oat cell cancer; melanoma including cutaneous melanoma and metastatic melanoma; mesothelioma; multiple myeloma including bone marrow cancer; neuroblastoma; neuroendocrine tumours; Non-Hodgkin lymphoma (NHL) including B-Cell lymphoma, lymph node cancer, lymphoma, mycosis fungoides and T-cell lymphoma; ocular cancer; ocular melanoma; oral cancer including lip cancer, oral cavity cancer, jaw cancer, kaposi sarcoma, mouth cancer, mucosal melanoma, salivary gland cancer and tongue cancer; ovarian cancer including fallopian tube cancer, ovarian epithelial cancer, ovarian germ cell tumour, ovarian primary peritoneal carcinoma, ovarian sex cord stromal tumour and peritoneal cancer; pancreatic cancer including islet cell cancer; paranasal sinus cancer; pelvic cancer; penile cancer; primary central nervous system lymphoma; prostate cancer; soft tissue sarcoma including fibrosarcoma and synovial sarcoma; sinus cancer; skin cancer including basal cell carcinoma, cutaneous lymphoma, squamous cell carcinoma and Merkel cell carcinoma; small intestine cancer; soft tissue sarcoma including angiosarcoma, epithelioid sarcoma, liposarcoma; leiomyosarcoma and rhabdomyosarcoma; spinal cancer including spinal column cancer, spinal cord cancer and spinal tumour; stomach cancer including carcinoid tumours and gastric cancer; testicular cancer; throat cancer including hypopharyngeal cancer, laryngeal cancer, nasal cavity cancer, nasopharyngeal cancer, oropharyngeal cancer and pharyngeal cancer; thymoma or thymic carcinoma; thyroid cancer including parathyroid cancer; tubal cancer; urethral cancer; uterine cancer including endometrial cancer, uterine adenocarcinoma, uterine sarcoma and uterine sarcoma; vaginal cancer and vulvar cancer. Most preferably the cancer is breast cancer, lung cancer, kidney cancer or pancreatic cancer.

In a second aspect of the invention there is provided for a kit for obtaining and culturing primary tumour cells from a tissue sample. Preferably, the kit comprises an isolation buffer which includes collagenase II; and a first propagation medium which includes ethanonamine and either estradiol or EGF; and further wherein the kit includes instructions for use instructing a user to digest the cells in the isolation buffer for a period of 45 minutes or less at about 37° C.

The kit may further comprise a second propagation medium wherein if the first propagation medium included estradiol, then the second propagation medium includes cortisol and does not include estradiol. Alternatively, if the first propagation medium included EGF, then the second propagation medium includes cortisol and EGF.

In one embodiment of the invention the first propagation medium and second propagation medium in the kit also include DMEM/F12, human A/B serum, an antibiotic, sodium pyruvate, insulin, transferrin, and/or selenium.

In a preferred embodiment of the invention the tissue sample is obtained from a tumour biopsy. Preferably, the tissue sample is obtained from a human.

### BRIEF DESCRIPTION OF THE FIGURES

Non-limiting embodiments of the invention will now be described by way of example only and with reference to the following figures:

FIG. 1: Morphological visualization of propagated primary breast cancer cells. The cells were visualized using an inverted light microscope at 5× magnification (Nikon). The images are representative of samples from four individual donors at days 0, 3 and 7. The primary breast cancer cells were successfully propagated for 3 months. Arrows indicate the adherent populations of cells.

FIG. 2: Morphological visualization of the primary lung cancer cells. The cells were visualized using an inverted light microscope at 5× magnification (Nikon). The images are representative of samples from two individual donors at day 7 (A) and day 14 (B). We have successfully propagated the primary lung cancer cells for several weeks. Black arrows indicate the adherent population of cells.

FIG. 3: Morphological visualization of the primary prostate and kidney cancer cells. The cells were visualized using an inverted light microscope at 10× magnification (Nikon). The images are representative of primary prostate (A) and kidney (B) cancer samples cultured at day 1 and 2. Black arrows indicate the adherent population of cells.

FIG. 4: Morphological visualization of the primary kidney cancer cells at day 5 and 7. The cells were visualized using an inverted light microscope at 10× magnification (Nikon). Black arrows indicate the adherent population of cells.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The present invention relates to an in vitro method of obtaining and culturing primary tumour cells from a tissue sample using an isolation buffer which includes collagenase II, and optionally hyaluronidase and a propagation medium which includes estradiol or EGF. The invention is further directed to a kit for obtaining and culturing primary tumour cells from a tissue sample.

The applicant has identified a need to develop a method and product in a kit-based format for the isolation and propagation of primary cancer cells because they represent an ideal in vivo model system.

Cell lines are extensively used in the majority of in vitro cancer studies because they are easy to maintain and grow in tissue culture. In many studies they have been important to the understanding of tumour biology. However, a number of these cell lines were produced a number of years ago and as a result many have undergone significant genomic and phenotypic drift. These genetically altered cancer cell lines have limited clinical use because they are not truly representative of an in vivo model. For this reason, we have developed a method and kit for isolating and propagating primary tumour cells for use in in vitro experiments, because they are more representative of an in vivo model.

The technology and methodology described herein will assist researchers and vaccine developers to easily isolate and propagate primary cancer cells from a particular subject.

The present inventors have shown that the method of the present invention is useful for successfully isolating, propagating and maintaining primary cancer cells on a regular basis.

The method and/or kit of the present invention uses collagenase II (protease) and optionally hyaluronidase to isolate the tumour cells and this is combined with a specially formulated growth medium containing growth factors, hormones, supplements, antibiotics and steroids to propagate the tumour cells.

The tumour cells are initially digested for a period of 45 minutes at 37° C. in the presence of collagenase II and optionally in the presence of either hyaluronidase, dispase II or papain. The cellular yield of cells digested in the presence of both collagenase II and hyaluronidase increased by 2.7-fold, whereas the cellular yield of cells digested in the presence of either collagenase II and dispase II or collagenase and papain increased by 0.7-fold.

The inventors have found that by using a relatively short time period for the digestion step of 45 minutes results in a higher yield of cells and an increase in cellular viability as compared to other methods which rely on longer periods for digestion. Other methods that use longer incubation periods and/or collagenase II in conjunction with trypsin generally have been found to have lower cellular yields as a result of proteolytic activity causing cell death, decreased cellular viability and decreased propagation of the primary cells. Further, longer incubation periods and/or the use of collagenase II in conjunction with trypsin results in changes in cellular morphology which could impact on the efficacy of the cells for use in a vaccine.

The method and/or kit of the present invention further includes a buffer that provides an ideal pH, ions and other factors which are required for optimal enzymatic activity of the enzyme.

The method and/or kit of the present invention includes an isolation buffer and a first propagation medium, wherein the isolation buffer includes collagenase II, and optionally hyaluronidase, papain or dispase II and the first propagation medium includes human A/B serum, penicillin or streptomycin, sodium pyruvate, insulin, transferrin, ethanolamine, selenium and estradiol or EGF.

The addition of ethanolamine to the propagation medium has extensive benefits. Firstly, ethanolamine is essential for phospholipid formation, which is an important component of the mammalian cell membrane. Cells grown in the absence of ethanolamine have been shown to have reduced phospholipid content (˜50%) in their cell membrane. Secondly, ethanolamine has also been shown to be an essential supplement for the proliferation of many mammalian cell types including epithelial, oesophageal, bronchial, and lung cells. There is further evidence to suggest that ethanolamine enhances DNA synthesis in some cell types and it is an important supplement for the long-term growth of cancer cells, which is important for vaccine development. Further it has been shown that cells grown in the absence of ethanolamine do not survive for extended periods in in vitro culture. Ethanolamine is essential to maintain cellular homeostasis, metabolic activity and proliferation. The inventors would expect that cells grown in the absence of ethanolamine would have very different growth characteristics, metabolic activity and longevity in in vitro culture.

Further, the addition of estradiol or EGF for use in the culture of breast, lung, pancreatic and kidney cancer cells leads to an increase in the proliferation of these cells in in vitro culture. Lung, pancreas and kidney cancer cells express epithelial growth factor receptor (EGFR), which is important for proliferation in these cells. On the other hand, breast cancer cells express the estrogen receptor, which is important for proliferation. Estradiol was added to the breast culture propagation medium in order to increase proliferation of the breast cancer cells in vitro.

The method and/or kit of the present invention also includes a second propagation medium, which includes human A/B serum, penicillin or streptomycin, sodium pyruvate, insulin, transferrin, ethanolamine and selenium, and wherein if the first propagation medium contained estradiol this is replaced with cortisol or if the first propagation medium contained EGF then the second propagation medium includes both EGF and cortisol.

The addition of cortisol to the second propagation medium prevents the proliferation or growth of fibroblasts in the cell culture allowing the epithelial cells in the culture to grow.

The isolation buffer of the present invention may also include one or more other protease enzymes, such as collagenase I, II, II, elastase, hyaluronidase, papain, and Dispase®.

The propagation medium of the present invention may also include other growth factors such as activin, colony stimulating factor, epidermal growth factor, fibroblast growth factor, insulin-like growth factor, stem cell factor and/or transforming growth factor; supplements such as amino acids and/or carbon sources; steroids such as dexamethasone, prednisolone and/or testosterone; antibiotics such as kanamycin, tetracycline and/or gentamycin; and hormones such as progesterone, luteinizing hormone, adrenalin, glucagon, oxytocin, thyroxine and/or vasopressin.

The term “cell culture” refers to maintenance and growth, cultivation, or expansion of cells dissociated from the parent tissue in an artificial environment outside of the host's body. This can be termed an in vitro environment. The use of the term “cell culture” is generic and can be used interchangeably with the term “tissue culture”. Both terms, “cell culture” and “tissue culture,” can be used when referring to individual cells, a group of cells, a group or mixture of different or like cell types, tissues, and organs.

The terms “propagation medium”, “cell culture medium,” “culture medium,” “tissue culture medium,” can be used interchangeably and refer to a nutritional solution for cultivating cells, tissues, or organs.

A “primary cell culture” refers to the cell culture initially derived from the parent tissue prior to any subsequent culture in vitro, or on a cell culture vessel. The cells may be isolated directly from samples of tissue obtained by biopsy, autopsy, surgical or medical procedure, donation, or harvesting. The cells attach and spread across the culture vessel, forming a monolayer of cells. Upon adequate growth and expansion, determined by one skilled in the art, the cells are dissociated from the vessel and, diluted into fresh culture vessels. This is known by those skilled in the art as passaging. Subsequent passaging of the primary cell culture yields an expanded culture of cells derived, or originating, from the original tissue.

The following example is offered by way of illustration and not by way of limitation.

### Example 1

Isolation and Propagation of Primary Breast Cancer Cells

Primary Cell Isolation

The method of the present invention has been successfully used to isolate primary breast cancer cells from 10-200 mg of tissue. A fresh tumour biopsy sample was placed in 5 ml Hanks Balanced Salts (HBSS; Lonza, Germany; pH=7.5) on ice and it was transported immediately to the laboratory. The tumour biopsy sample was weighed and then cut into 1 mm by 1 mm pieces using a scalpel in a BSL2 cabinet. The cut pieces were placed in 1 ml HBSS containing 5 μg/ml collagenase II (Ambion, USA) and the tissue was digested by incubation at 37° C. with rotation for 45 min. After the incubation time, a 0.7 μM cell strainer (Becton Dickinson, USA) was placed into a 50 ml tube, and the cellular suspension was placed onto the cell strainer. The cell strainer was washed with 10 ml of HBSS and the cells were pelleted at 500×g for 5 min. The pelleted cells were washed with an additional 10 ml of HBSS.

Propagation of the Primary Cells

The pelleted cells were re-suspended in DMEM/F12 containing 10% human A/B serum (Western Province Blood Transfusion Services), 100 IU penicillin/streptomycin (Lonza, Germany), 0.1 mM sodium pyruvate (Lonza, Germany), 5 mg/ml insulin (Sigma, Germany), 5 mg/ml transferrin (Sigma, Germany), 5 mM ethanolamine (Sigma, Germany), 5 μg/ml selenium (Sigma, Germany) and 10 nM estradiol (Sigma, Germany). The cells were plated into a multi well plate or dish, which was dependent on the cell number. After 2 days of incubation at 37° C. the medium was replaced with fresh medium without 10 nM estradiol, but with 100 nM cortisol (Sigma, Germany). Cortisol was added to the medium to prevent fibroblast growth. The medium was replenished every 2 days with fresh medium containing half the concentration of cortisol used previously. When the cells were confluent they were split using a standard procedure, using trypsin, into a larger culture vessel. The cells were cultured until the medium did not contain cortisol.

Results

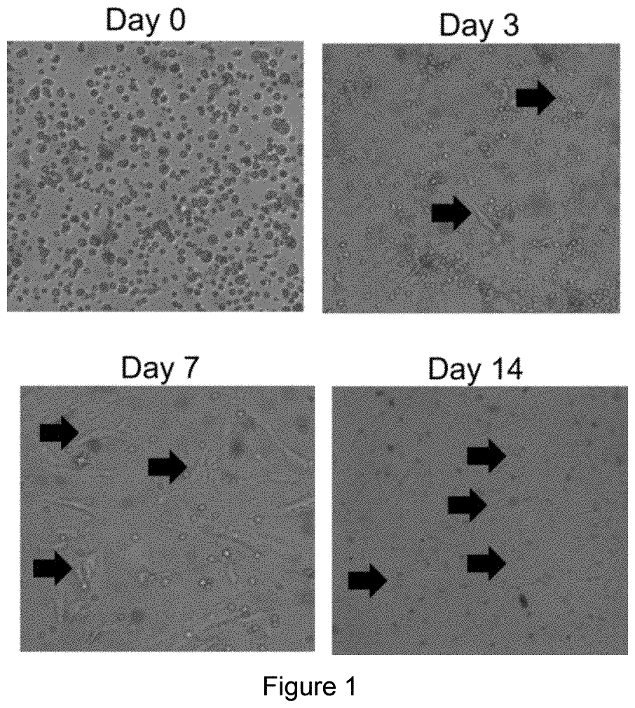

Preliminary data indicates that primary breast cancer cells were propagated from tumour biopsies obtained from individual patients (FIG. 1). At day 1 the primary epithelial cells were non-adherent, but became more adherent over time. The applicant has managed to culture the cells for several months in the laboratory. Data from the breast cancer preclinical trial show that the present inventors could reproducibly isolate primary breast cancer cells from breast biopsies and keep these cells in culture for several months in the laboratory.

### Example 2

Optimisation of the Method for Isolation and Propagation of Primary Breast Cancer Cells

Primary Cell Isolation

A fresh primary breast cancer tumour biopsy sample (10-200 mg of tissue) was placed in 5 ml Hanks Balanced Salts (HBSS; Lonza, Germany; pH=7.5) on ice and it was transported immediately to the laboratory. The biopsy sample was weighed and then cut into 1 mm by 1 mm pieces using a scalpel in a BSL2 cabinet. The cut pieces were placed in 1 ml HBSS containing 5 μg/ml collagenase II (Ambion, USA) with or without 5 U/ml hyaluronidase, dispase II or papain (10 mg/ml) and the tissue was digested by incubation at 37° C. with rotation for 45 min. After the incubation time, a 0.7 μM cell strainer (Becton Dickinson, USA) was placed into a 50 ml tube. The undigested material was pipetted up and down 10 times and then placed onto the cell strainer. The cell strainer was washed with 10 ml of HBSS and the cells were pelleted at 500×g for 5 min. The pelleted cells were then washed with an additional 10 ml of HBSS.

Propagation of the Primary Cells

The pelleted cells were re-suspended in DMEM/F12 containing 10% human A/B serum (Western Province Blood Transfusion Services), 100 IU penicillin/streptomycin (Lonza, Germany), 0.1 mM sodium pyruvate (Lonza, Germany), 5 mg/ml insulin (Sigma, Germany), 5 mg/ml transferrin (Sigma, Germany), 5 mM ethanolamine (Sigma, Germany), 5 μg/ml selenium (Sigma, Germany) and 10 nM estradiol (Sigma, Germany). The cells were then plated into a multi well plate or dish, which was dependent on the cell number. The breast cancer cells were incubated at 37° C. for 2 days and the medium was replaced with fresh medium without 10 nM estradiol, but with 100 nM cortisol (Sigma, Germany). The medium was replaced every 2 days with fresh medium containing half the concentration of cortisol used on the previous day. When the cells were confluent they were split using a standard procedure (trypsin) into a larger culture vessel.

Results

The data indicated that when hyaluronidase was added with collagenase II the cellular yield improved 2.7-fold, compared to cells isolated from the biopsy samples using collagenase II alone (Table 1). The addition of dispase or papain together with collagenase II did not improve the yield of cells (0.7-fold) from the biopsy samples compared to cells isolated from the biopsy samples with collagenase II alone. The mean viability of the cells was 69% and 84% when hyaluronidase and dispase were used in conjunction with collagenase II, respectively. Overall the results indicate that the addition of hyaluronidase significantly improves the yield of cells from the biopsy samples.

TABLE 1
Hyaluronidase, but not dispase or papain, used in conjunction with
collagenase II improves breast cancer primary cell yield from breast
biopsy samples above that observed with collagenase II only.

| Additional enzyme used in | Mean fold increase | |
|---|---|---|
| conjunction with collagenase II | in cell number | Mean cell |
| for cell isolation | (collagenase II = 1) | viability |
| Hyaluronidase (n = 4) | 2.7 (SD = 1.2-4.5) | 69 (SD = 33-81) |
| Dispase (n = 3) | 0.7 (SD = 0.2-1.1) | 84 (SD = 75-92) |
| Papain (n = 3) | 0.7 (SD = 0.4-0.9) | *ND |

*ND = not determined because of low cell numbers

### Example 3

Optimisation of the Method for Isolation and Propagation of Primary Lung, Kidney and Pancreatic Cancer Cells

Primary Cell Isolation

A fresh lung, kidney or pancreas cancer biopsy sample (10-200 mg of tissue) was placed in 5 ml Hanks Balanced Salts (HBSS; Lonza, Germany) on ice and transported immediately to the laboratory. The biopsy sample was weighed and then cut into 1 mm by 1 mm pieces using a scalpel in a BSL2 cabinet. The cut pieces were placed in 1 ml HBSS containing 5 μg/ml collagenase II (Ambion, USA) with or without 5 U/ml hyaluronidase, dispase II or papain (10 mg/ml) and the tissue was digested by incubation at 37° C. with rotation for 45 min. After incubation, a 0.7 μM cell strainer (Becton Dickinson, USA) was placed into a 50 ml tube. The undigested material was pipetted up and down 10 times and then placed onto the cell strainer. The cell strainer was washed with 10 ml of HBSS and the cells were pelleted at 500×g for 5 min. The pelleted cells were then washed with an additional 10 ml of HBSS.

Propagation of the Primary Cells

The pelleted cells were re-suspended in DMEM/F12 containing 10% human A/B serum (Western Province Blood Transfusion Services), 100 IU penicillin/streptomycin (Lonza, Germany), 0.1 mM sodium pyruvate (Lonza, Germany), 5 mg/ml insulin (Sigma, Germany), 5 mg/ml transferrin (Sigma, Germany), 5 mM ethanolamine (Sigma, Germany), 5 μg/ml selenium (Sigma, Germany) and 10 ng/ml epithelial growth factor (EGF) (Sigma, Germany). The cells were then plated into a multi well plate or dish, which was dependent on the cell number. The lung, kidney or pancreatic cancer cells were incubated at 37° C. for 2 days and the medium was replaced with fresh medium containing 10 ng/ml EGF and 100 nM cortisol. The medium was replaced every 2 days with fresh medium containing half the concentration of cortisol used on the previous day. When the cells were confluent they were split using a standard procedure (trypsin) into a larger culture vessel.

Results

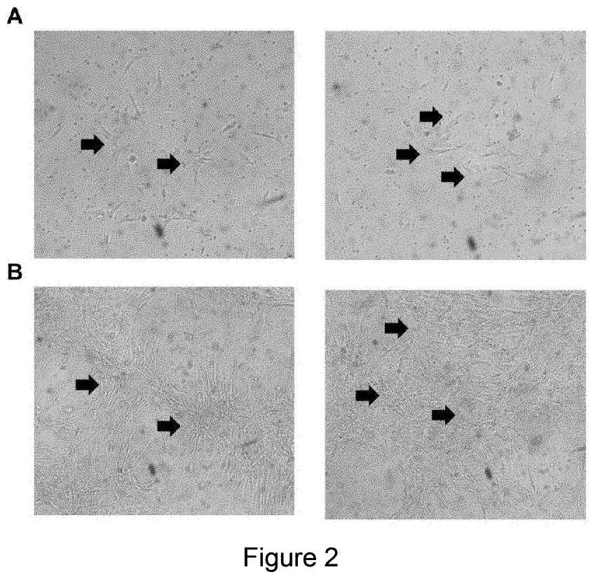

The present inventors successfully isolated and propagated autologous lung cancer cells, which could be culture for a number of weeks (FIG. 2). Using flow cytometry, it was shown that the cells expressed the epithelial marker Ep-CAM and the progenitor marker CD49f (data not shown). The data thus indicates that the method of isolation and propagation disclosed herein has a wider application to a number of different cancers.

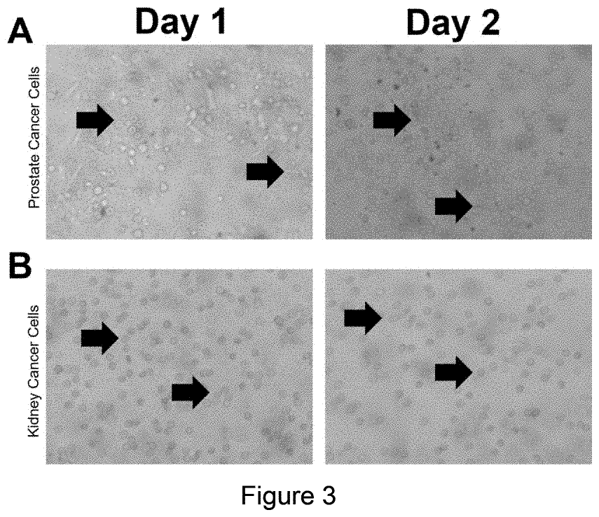

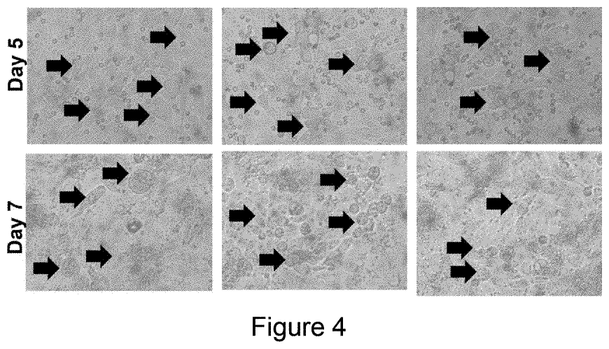

Having shown that the methodology is successful at isolating and propagating breast and lung cancer cells, the inventors applied the methodology to pancreatic and kidney cancer cells. Successful isolation and propagation of primary pancreatic (FIG. 3A) and kidney cancer (FIG. 3B) cells from biopsy samples are shown in FIG. 3. The kidney cancer cell morphology changed by day 5 and 7, the cells were confluent, and they were split into a larger culture vessel (FIG. 4).

1. An in vitro method of obtaining and culturing primary tumour cells from a tissue sample, the method comprising the steps of:
i. isolating primary tumour cells using an isolation buffer including collagenase II for a period of 45 minutes or less at about 37° C.; and
ii. propagating the cells obtained in step i) using a first propagation medium including:
a) estradiol or EGF; and
b) ethanolamine.

2. The in vitro method of claim 1, further comprising a step of propagating the cells in a second propagation medium, wherein:
a) if the first propagation medium included estradiol, replacing the estradiol with cortisol; or
b) if the first propagation medium included EGF adding cortisol to the second propagation medium.

3. The in vitro method of claim 2, wherein the first propagation medium and second propagation medium further include DMEM/F12, human A/B serum, an antibiotic, sodium pyruvate, insulin, transferrin, and/or selenium.

4. The in vitro method of claim 3, wherein the first propagation medium and second propagation medium optionally includes hyaluronidase, dispase II or papain.

5. The in vitro method of claim 3, wherein the antibiotic is penicillin or streptomycin.

6. The in vitro method of claim 1, further comprising a step of splitting the cells using trypsin.

7. The in vitro method of claim 1, wherein the tissue sample is obtained from a tumour biopsy.

8. The in vitro method of claim 1, wherein the tissue sample is obtained from a human.

9. A kit for obtaining and culturing primary tumour cells from a tissue sample, wherein the kit comprises:
i. an isolation buffer including collagenase II;
ii. a first propagation medium including ethanolamine and either estradiol or EGF, and
iii. instructions for use, wherein the instructions for use instruct a user to digest the cells in the isolation buffer for a period of 45 minutes or less at about 37° C.

10. The kit of claim 9, further comprising a second propagation medium wherein:
a) if the first propagation medium includes estradiol, replacing the estradiol with cortisol; or
b) if the first propagation medium includes EGF, adding cortisol to the second propagation medium.

11. The kit of claim 10, wherein the first propagation medium and second propagation medium further include DMEM/F12, human A/B serum, an antibiotic, sodium pyruvate, insulin, transferrin, and/or selenium.

12. The kit of claim 11, wherein the first propagation medium and second propagation medium optionally include hyaluronidase, dispase II or papain.

13. The kit of claim 11, wherein the antibiotic is penicillin or streptomycin.

14. The kit of claim 9, wherein the tissue sample is obtained from a tumour biopsy.

15. The kit of claim 9, wherein the tissue sample is obtained from a human.
